(19) United States
(12) Patent Application Publication
(10) Pub. No.: US 2026/0146056 A1
(43) Pub. Date: May 28, 2026

(54) MORPHOLINE-BASED ORGANOPHOSPHINE LIGAND, PREPARATION METHOD THEREFOR AND APPLICATION THEREOF

(71) Applicants: Henan Academy of Sciences Institute of Chemistry Co., Ltd, Zhengzhou (CN); Henan Academy of Sciences, Zhengzhou (CN)

(72) Inventors: Tingting Liu, Zhengzhou (CN); Xilong Wang, Zhengzhou (CN); Hui Chen, Zhengzhou (CN); Duo Zhou, Zhengzhou (CN); Lin Hua, Zhengzhou (CN); Jingyi Zhang, Zhengzhou (CN); Linlu Li, Zhengzhou (CN); Zhiyi Song, Zhengzhou (CN); Shumin Chen, Zhengzhou (CN); Fengbo Qu, Zhengzhou (CN)

(21) Appl. No.: 19/403,176

(22) Filed: Nov. 27, 2025

(30) Foreign Application Priority Data
Nov. 28, 2024 (CN) 202411730419.2

(51) Int. Cl. C07F 9/6533; C07C 209/68

(52) U.S. Cl. CPC C07F 9/6533; C07C 209/68

(57) ABSTRACT

A preparation method for a morpholine-based organophosphine ligand includes the steps of: adding 1,3-dibromobenzene, morpholine, an inorganic base, and a first catalyst to a reactor under a nitrogen (N₂) atmosphere, and using tetrahydrofuran (THF) as a reaction solvent; reacting a mixture at 45-55° C. for 5-7 h; lowering a reaction temperature to −75 to −80° C., and adding n-butyllithium (nBuLi); and raising the reaction temperature to 0° C. after stirring, and adding a second catalyst and a halophosphine reagent for reaction to obtain a morpholine-based organophosphine ligand. A method for synthesizing novel morpholine-based organophosphine ligands under mild conditions of the present disclosure has been successfully applied in palladium (Pd)-catalyzed carbon-nitrogen (C—N) bond formation reactions, demonstrating high efficiency and selectivity, simplifying reaction conditions, and broadening the scope of application.

### CROSS-REFERENCE TO RELATED APPLICATION

This application claims priority of Chinese Patent Application No. 202411730419.2, filed on Nov. 28, 2024, the entire contents of which are incorporated herein by reference.

### TECHNICAL FIELD

The present disclosure belongs to the technical field of organic phosphine ligands, and specifically relates to a morpholine-based organophosphine ligand, a preparation method therefor and an application thereof.

### BACKGROUND

For the palladium (Pd)-catalyzed cross-coupling reaction of haloarenes with amines, pioneering and systematic studies by researchers such as Buchwald and Hartwig have established an efficient method for the synthesis of N-substituted aromatic amines. This type of reaction is known as the Buchwald-Hartwig cross-coupling reaction. In this reaction, bulky and strongly electron-donating monodentate or bidentate phosphine ligands, such as tri-tert-butylphosphine (P(t-Bu)₃), tricyclohexylphosphine (PCy₃), 2-di-tert-butylphosphino-2′,4′,6′-triisopropylbiphenyl (t-Butyl-XPhos), and 2-dicyclohexylphosphino-2′-(N,N-dimethylamino) biphenyl (Dave Phos), are commonly employed. These ligands can cause the Pd-catalyzed carbon-nitrogen (C—N) bond formation to proceed efficiently under mild conditions. However, the aforementioned phosphine ligands often exhibit poor stability or present challenges for straightforward synthesis. Consequently, exploration of novel ligands and corresponding synthesis methods is highly necessitated.

Morpholine is an important class of N-containing heterocyclic compounds with broad application value and significance in pharmaceutical research and development, as well as organic synthesis. Given the promising application prospects of morpholine-based compounds, novel morpholine derivatives, particularly those integrated with organophosphine ligands, have attracted considerable attention from chemists and pharmacologists. This class of ligands possesses the electron-rich nature of organophosphine ligands while featuring the defined steric framework of the morpholine structure. Therefore, it is a significant research direction in organic synthesis to explore an efficient synthesis method under mild conditions and apply it to catalytic C—N bond construction.

### SUMMARY

An objective of the present disclosure is to provide a morpholine-based organophosphine ligand, a preparation method therefor and an application thereof, aiming to solve the problems of poor stability and relatively complex synthesis methods associated with the conventional organophosphine ligands used for catalytic C—N bond formation.

To realize the above objective, a first aspect of the present disclosure provides a morpholine-based organophosphine ligand, having a general chemical formula:

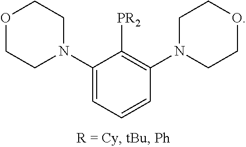

A second aspect of the present disclosure provides a preparation method for the morpholine-based organophosphine ligand, including the steps of:
adding 1,3-dibromobenzene, morpholine, an inorganic base, and a first catalyst to a reactor under a nitrogen (N₂) atmosphere, and using tetrahydrofuran (THF) as a reaction solvent; reacting a mixture at 45-55° C. for 5-7 h; lowering a reaction temperature to −75 to −80° C., and adding n-butyllithium (nBuLi); and raising the reaction temperature to 0° C. after stirring, and adding a second catalyst and a halophosphine reagent for reaction to obtain a morpholine-based organophosphine ligand.

A chemical reaction formula of the present disclosure is:

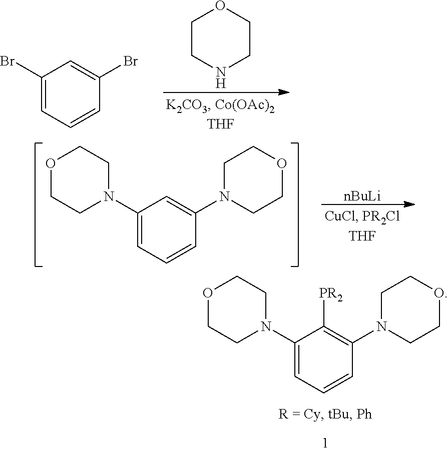

Preferably, the inorganic base is potassium carbonate (K₂CO₃), and the first catalyst is cobalt acetate (Co(OAc)₂).

Preferably, a mass ratio of 1,3-dibromobenzene, morpholine, the inorganic base, and the first catalyst is 10-25 g: 15-20 g: 32-35 g: 1.5-2 g.

Preferably, nBuLi, at a concentration of 1.5-1.8 M, is added in a volume of 60-65 mL.

Preferably, the second catalyst is copper (I) chloride (CuCl), and a mass ratio of CuCl to the halophosphine reagent is 0.8-1.0 g: 20-25 g.

A third aspect of the present disclosure provides an application of the morpholine-based organophosphine ligand in a cross-coupling reaction between a haloarene and an amine compound.

Preferably, the cross-coupling reaction between the haloarene and the amine compound includes the steps of:
adding 4-Bromotoluene, a base, a third catalyst, and the morpholine-based organophosphine ligand to a reactor; purging the reactor with N₂ for three times, and adding toluene and aniline; sealing the reactor, and placing the sealed reactor into a preheated oil bath at 95-105° C.; and heating a mixture with stirring to obtain a target product, 4-methyl-N-phenylaniline.

Preferably, the base is sodium tert-butoxide (NaOtBu), and the third catalyst is tris(dibenzylideneacetone)dipalladium (Pd₂(dba)₃).

Preferably, a molar ratio of 4-Bromotoluene, the base, the third catalyst, the morpholine-based organophosphine ligand, and aniline is 0.8-1.2:1.2-1.6:0.01-0.015:0.04-0.08:1.0-1.4.

Therefore, the present disclosure employing the morpholine-based organophosphine ligand with the aforementioned structure, the preparation method and the application, provides the following beneficial effects.

1. A synthesis pathway of the present disclosure is novel. By utilizing starting materials such as 1,3-dibromobenzene and morpholine, a target ligand is synthesized through multi-step reactions under specific conditions, providing a novel and efficient synthetic route.

2. Reaction conditions of the present disclosure are mild. The entire synthesis process employs temperature ranges from room temperature to 55° C., as well as cryogenic conditions of −75° C. to −80° C. These conditions are relatively mild and help minimize the occurrence of side reactions.

3. A process of the present disclosure is operationally simple. The solvents (e.g., THF) and reagents (e.g., nBuLi) used in the synthesis are all common chemicals, readily available. The procedural steps are also straightforward, facilitating large-scale production.

4. The organophosphine ligand prepared by the present disclosure can enhance catalytic efficiency. When employed as a ligand in the Pd-catalyzed cross-coupling reaction between the haloarene and the amine compound, it can demonstrate superior catalytic activity or selectivity compared to traditional phosphine ligands.

5. The organophosphine ligand of the present disclosure combines the characteristics of the morpholine and the organophosphine. This class of ligands not only facilitates the C—N bond formation but is also potentially applicable to other types of coupling reactions, thereby increasing its application versatility.

6. The organophosphine ligand prepared by the present disclosure for C—N bond synthesis is used for C—N bond synthesis via a coupling reaction conducted at 95-105° C., thereby facilitating relatively straightforward control during industrial scale-up and contributing to cost reduction. A C—N bond synthesis reaction system of the present disclosure employs simple bases such as NaOtBu and established catalysts such as Pd₂(dba)₃, thereby simplifying the reaction conditions and enhancing the feasibility of the reaction.

The technical solutions of the present disclosure are further described in detail with reference to the embodiments.

### DETAILED DESCRIPTION

The present disclosure is further described below. It is to be noted that this embodiment is based on the technical solutions described herein, providing detailed implementation methods and specific operating procedures. However, the present disclosure is not limited to this embodiment.

### Embodiment 1

A preparation method for a morpholine-based organophosphine ligand includes the following steps:

Under N₂ atmosphere, 1,3-dibromobenzene (23.5 g), morpholine (18.3 g), K₂CO₃ (34.5 g), and Co(OAc)₂ (1.7 g) were added to a dried reactor using THF as a reaction solvent. A mixture was reacted at 50° C. for 6 h. The reaction temperature was lowered to −78° C., and 1.6 M nBuLi (62.5 mL) was added, followed by stirring for 1 h. A reaction system was warmed to 0° C. and reacted with 23.2 g of chlorocyclohexylphosphine in the presence of CuCl (0.99 g) as a catalyst, to obtain a product, 4,4′-(2-(Dicyclohexylphosphanyl)-1,3-phenylene)dimorpholine (34.6 g, 78% yield).

Proton nuclear magnetic resonance spectroscopy (¹H NMR) and carbon-13 nuclear magnetic resonance spectroscopy (¹³C NMR) data for the prepared product are provided below, confirming the successful synthesis of this product.

¹H NMR (400 MHz, CDCl₃) δ 6.96 (t, J=15.0 Hz, 1H), 6.07 (d, J=15.0 Hz, 2H), 3.72 (t, J=9.2 Hz, 8H), 3.18 (t, J=9.1 Hz, 8H), 2.21-1.88 (m, 4H), 1.68-1.50 (m, 6H), 1.33-1.01 (m, 10H). ¹³C NMR (100 MHz, CDCl₃) δ 166.7, 153.8, 131.9, 112.5, 65.8, 47.4, 29.5, 25.9, 25.6, 23.0.

### Embodiment 2

A preparation method for a morpholine-based organophosphine ligand includes the following steps:

Under N₂ atmosphere, 1,3-dibromobenzene (23.5 g), morpholine (18.3 g), K₂CO₃ (34.5 g), and catalyst Co(OAc)₂ (1.7 g) were added to a dried reactor using THE as a reaction solvent. A mixture was reacted at 50° C. for 6 h. The reaction temperature was lowered to −78° C., and 1.6 M nBuLi (62.5 mL) was added, followed by stirring for 1 h. A reaction system was warmed to 0° C. and reacted with chlorodiphenylphosphine (22.1 g) in the presence of CuCl (0.99 g) as a catalyst, to obtain a product, 4,4′-(2-(Diphenylphosphanyl)-1,3-phenylene)dimorpholine (31.5 g, 73% yield).

¹H NMR and ¹³C NMR data for the prepared product are provided below, confirming the successful synthesis of this product.

¹H NMR (400 MHz, CDCl₃) δ 7.52-7.40 (m, 4H), 7.40-7.25 (m, 6H), 7.00 (t, J=15.0 Hz, 1H), 6.08 (d, J=14.9 Hz, 2H), 3.71 (t, J=9.2 Hz, 8H), 3.17 (t, J=9.2 Hz, 8H). ¹³C NMR (100 MHz, CDCl₃) δ 156.2, 138.9, 134.0, 132.8, 129.2, 128.6, 123.3, 115.0, 65.8, 47.4.

### Embodiment 3

A preparation method for a morpholine-based organophosphine ligand includes the following steps:

Under N₂ atmosphere, 1,3-dibromobenzene (23.5 g), morpholine (18.3 g), K₂CO₃ (34.5 g), and catalyst Co(OAc)₂ (1.7 g) were added to a dried reactor using THE as a reaction solvent. A mixture was reacted at 50° C. for 6 h. The reaction temperature was lowered to −78° C., and 1.6 M nBuLi (62.5 mL) was added, followed by stirring for 1 h. A reaction system was warmed to 0° C. and reacted with tert-butylchlorophosphine (23.1 g) in the presence of CuCl (0.99 g) as a catalyst, to obtain a product, 4,4′-(2-(dicyclohexylphosphanyl)-1,3-phenylene)dimorpholine.

¹H NMR and ¹³C NMR data for the prepared product are provided below, confirming the successful synthesis of this product.

¹H NMR (400 MHz, CDCl₃) δ 6.96 (s, 1H), 6.07 (d, J=14.9 Hz, 2H), 3.72 (t, J=9.2 Hz, 8H), 3.18 (t, J=9.2 Hz, 8H), 1.15 (s, 18H). ¹³C NMR (100 MHz, CDCl₃) δ 170.8, 170.6, 153.8, 153.7, 132.5, 132.5, 106.3, 106.3, 65.9, 49.8, 49.6, 47.4, 47.4, 28.0, 27.9.

### Embodiment 4

The morpholine-based organophosphine ligands prepared in Embodiments 1-3 were applied to C—N bond formation reactions, with a specific application procedure as follows:

4-Bromotoluene (171 mg, 1.0 mmol), NaOtBu (135 mg, 1.4 mmol), Pd₂(dba)₃ (9.2 mg, 0.01 mmol), and the morpholine-based organophosphine ligands from Embodiments 1-3 (0.06 mmol) were added to a 25 mL Schlenk tube. The Schlenk tube was purged with N₂ for three times, followed by the addition of toluene (2.5 mL) and aniline (111.6 mg, 1.2 mmol). The Schlenk tube was sealed, and the sealed Schlenk tube was placed into a preheated oil bath at 100° C. and heated with stirring for 6 h. After completion, a reaction mixture was subjected to work-up procedures to obtain a target product, 4-methyl-N-phenylaniline.

### Comparative Embodiment 1

This comparative embodiment differs from Embodiment 4 only in the use of P(t-Bu)₃ instead of the morpholine-based organophosphine ligand. A chemical structure of P(t-Bu)₃ is represented as follows:

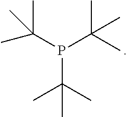

### Comparative Embodiment 2

This comparative embodiment differs from Embodiment 4 only in the use of PCy₃ instead of the morpholine-based organophosphine ligand. A chemical structure of PCy₃ is represented as follows:

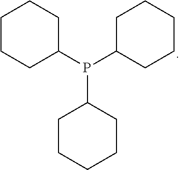 [SMILES: C1CCC(P(C2CCCCC2)C2CCCCC2)CC1]

### Comparative Embodiment 3

This comparative embodiment differs from Embodiment 4 only in the use of t-Butyl-XPhos instead of the morpholine-based organophosphine ligand. A chemical structure of t-Butyl-XPhos is represented as follows:

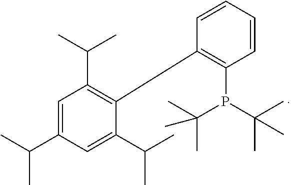 [SMILES: CC(C)c1cc(C(C)C)c(-c2ccccc2P(C(C)(C)C)C(C)(C)C)c(C(C)C)c1]

### Comparative Embodiment 4

This comparative embodiment differs from Embodiment 4 only in the use of Dave Phos instead of the morpholine-based organophosphine ligand.

A chemical structure of Dave Phos is represented as follows:

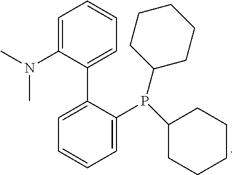 [SMILES: CN(C)c1ccccc1-c1ccccc1P(C1CCCCC1)C1CCCCC1]

The organophosphine ligands from Embodiments 1-3 and Comparative Embodiments 1-4 were placed under ambient air at room temperature for 90 days, and evaluated in the C—N bond formation reaction for the preparation of 4-methyl-N-phenylaniline according to the procedure in Embodiment 4. A yield of 4-methyl-N-phenylaniline is presented in Table 1.

TABLE 1

| | | Yield of 4-methyl- |
|---|---|---|
| | Initial yield | N-phenylaniline |
| | of 4-methyl-N- | after 90 days |
| Group | phenylaniline (%) | of storage (%) |
| Embodiment 1 | 95 | 92 |
| Embodiment 2 | 92 | 90 |
| Embodiment 3 | 94 | 91 |
| Comparative | 89 | 75 |
| Embodiment 1 | | |
| Comparative | 85 | 68 |
| Embodiment 2 | | |
| Comparative | 87 | 80 |
| Embodiment 3 | | |
| Comparative | 90 | 78 |
| Embodiment 4 | | |

Therefore, the present disclosure utilizes the aforementioned morpholine-based organophosphine ligand, the preparation method therefor and the application thereof, to provide a method for synthesizing novel morpholine-based organophosphine ligands under mild conditions. This method has been successfully applied in Pd-catalyzed C—N bond formation reactions, demonstrating high efficiency and selectivity, simplifying reaction conditions, and broadening the scope of application.

Finally, it is to be noted that the aforementioned embodiments are only used for stating the technical solutions of the present disclosure, rather than limiting the technical solutions. Although the present disclosure has been described in detail with reference to the preferred embodiments, it is to be understood by those of ordinary skill in the art that modifications or equivalent replacements to the technical solutions of the present disclosure may still be made without departing from the spirit and scope of the technical solutions of the present disclosure.

1. A morpholine-based organophosphine ligand, having a general chemical formula:

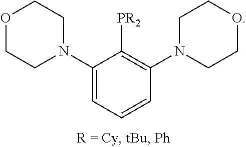

2. A preparation method for the morpholine-based organophosphine ligand according to claim 1, comprising the steps of:
adding 1,3-dibromobenzene, morpholine, an inorganic base, and a first catalyst to a reactor under a nitrogen (N₂) atmosphere, and using tetrahydrofuran (THF) as a reaction solvent; reacting a mixture at 45-55° C. for 5-7 h; lowering a reaction temperature to −75 to −80° C., and adding n-butyllithium (nBuLi); and raising the reaction temperature to 0° C. after stirring, and adding a second catalyst and a halophosphine reagent for reaction to obtain a morpholine-based organophosphine ligand; wherein
the inorganic base is potassium carbonate (K₂CO₃), the first catalyst is cobalt acetate (Co(OAc)₂), and the second catalyst is copper (I) chloride (CuCl).

3. The preparation method for the morpholine-based organophosphine ligand according to claim 2, wherein a mass ratio of 1,3-dibromobenzene, morpholine, the inorganic base, and the first catalyst is 10-25 g: 15-20 g: 32-35 g: 1.5-2 g.

4. The preparation method for the morpholine-based organophosphine ligand according to claim 3, wherein nBuLi, at a concentration of 1.5-1.8 M, is added in a volume of 60-65 mL.

5. The preparation method for the morpholine-based organophosphine ligand according to claim 4, wherein a mass ratio of CuCl to the halophosphine reagent is 0.8-1.0 g: 20-25 g.

6. An application of the morpholine-based organophosphine ligand according to claim 1 in a cross-coupling reaction between a haloarene and an amine compound.

7. The application of the morpholine-based organophosphine ligand according to claim 6, wherein the cross-coupling reaction between the haloarene and the amine compound comprises the steps of:
adding 4-Bromotoluene, a base, a third catalyst, and the morpholine-based organophosphine ligand to a reactor; purging the reactor with N₂ for three times, and adding toluene and aniline;
sealing the reactor, and placing the sealed reactor into a preheated oil bath at 95-105° C.; and
heating a mixture with stirring to obtain a target product, 4-methyl-N-phenylaniline.

8. The application of the morpholine-based organophosphine ligand according to claim 7, wherein the base is sodium tert-butoxide (NaOtBu), and the third catalyst is tris(dibenzylideneacetone)dipalladium (Pd₂(dba)₃).

9. The application of the morpholine-based organophosphine ligand according to claim 7, wherein a molar ratio of 4-Bromotoluene, the base, the third catalyst, the morpholine-based organophosphine ligand, and aniline is 0.8-1.2:1.2-1.6:0.01-0.015:0.04-0.08:1.0-1.4.
